(19) United States
(12) Patent Application Publication
(10) Pub. No.: US 2003/0022220 A1
(43) Pub. Date: Jan. 30, 2003

(54) Methods and compositions for modulating adenosine triphosphate (ATP) in cells and preventing cell injury or death via post-translational modifications to ATP synthase

(76) Inventors: Jennifer E. Van Eyk, Kingston (CA); David Kent Arrell, Kingston (CA)

Correspondence Address: Licata & Tyrrell P.C., 66 E. Main Street, Marlton, NJ, 08053, US

(21) Appl. No.: 10/189,820

(22) Filed: Jul. 3, 2002

Related U.S. Application Data
(60) Provisional application No. 60/303,491, filed on Jul. 6, 2001.

(51) Int. Cl. C12Q 1/68; C12Q 1/42

(52) U.S. Cl. 435/6; 435/21

(57) ABSTRACT

Compositions and methods for modulating adenosine triphosphate (ATP) in cells via altering post-translational modifications of ATP synthase subunits or precursors thereof such as the ATP synthase β chain and its precursor are provided. These compositions and methods are useful in preconditioning organs and preventing cell injury or cell death via regulating ATP synthesis or hydrolysis in cells of the organs.

### INTRODUCTION

This application claims the benefit of priority from U.S. Provisional Application Serial No. 60/303,491, filed Jul. 6, 2001, which is herein incorporated by reference in its entirety.

### BACKGROUND OF THE INVENTION

The adenosine triphosphate (ATP) synthase β chain is part of a multi-protein complex, referred to as ATP synthase or F₁Fₒ ATPase, located in the inner mitochondrial membrane. This complex catalyzes the final step in the oxidative phosphorylation process, wherein a hydrogen (H⁺) ion pump (Fₒ) is linked to ATP synthase (F₁). Directional flow through Fₒ is dependent upon the H⁺ ion concentration gradient across the inner mitochondrial membrane, and Fₒ in turn controls directional rotation and activity of the F₁ subunit. Under normal physiological conditions, H⁺ ions enter the mitochondrial matrix through Fₒ, and F₁ then synthesizes ATP. ATP is the fuel required for many energy-dependent intracellular processes, such as enzymatic activities, muscle contraction, second messenger signaling, and activation/inactivation of many membrane channels. During ischemia, however, F₁Fₒ activity is altered by a reduction of pH within the mitochondrial matrix. This reverses H⁺ ion flow through Fₒ, which in turn reverses F₁ rotation, resulting in ATP hydrolysis as opposed to synthesis.

ATP synthase has an unusual characteristic in that not all the protein subunits are encoded by a single genome. Some of the protein subunits are mitochondrial in origin, while others are encoded by the nuclear genome. The ATP synthase β chain is encoded by the nuclear genome. ATP synthase β chain is thus synthesized outside the mitochondria as a precursor, and must traverse the cytoplasm prior to mitochondrial entry and assembly into the mature ATP synthase complex. A portion of ATP synthase β chain precursor functions as a mitochondrial signaling peptide, which allows it to be taken up by mitochondria, and is removed from the mature protein during entry into mitochondria. There is evidence to indicate that there may also be turnover of the ATP synthase β chain precursor in the cytoplasm. It has been suggested that the ATP synthase β chain precursor may be phosphorylated, rendering the protein precursor less stable as indicated by an increase in proteolysis (Steinberg, R. A. J. Cell Biol. 1984 98(6):2174-8). To date, no other modifications of ATP synthase have been reported. Thus, the amount of protein available for incorporation into the mitochondria to form the ATP synthase complex appears to be strictly regulated, as is the amount of ATP generated by the cell at any given point in time.

ATP synthase beta subunit has been sequenced for four mammalian species (human, bovine, rat, and mouse), and it is a very highly conserved protein. The MW and pI for each (precursor and mature protein) is as follows:

| | | Precursor | | Mature Protein | |
|---|---|---|---|---|---|
| Species | Accession # | pI | MW | pI | MW |
| Human | P06576 | 5.26 | 56559.90 | 5.00 | 51769.25 |
| Bovine | P00829 | 5.15 | 56283.53 | 5.00 | 51562.97 |
| Rat | P10719 | 5.18 | 56353.55 | 4.95 | 51710.12 |
| Mouse | P56480 | 5.19 | 56300.49 | 4.99 | 51749.20 |

Thus, precursor is ˜56.3-56.6 kDa with pI ˜5.1-5.3 mature protein is ˜51.5-51.8 kDa with pI ˜4.9-5.0

### SUMMARY OF THE INVENTION

It has now been found that post-translational modifications of ATP synthase subunits and/or precursors thereof, in particular the ATP synthase β chain and its precursor, occur during pharmacological preconditioning, a treatment which mimics many aspects of classical ischemic preconditioning or hypoxia including protection of an organ from damage resulting from prolonged periods of ischemia, hypoxia, ischemia/reperfusion or any other event or agent that causes or promotes cell death (necrosis or apoptosis) or injury.

An aspect of the present invention relates to compositions and methods for modulating adenosine triphosphate (ATP) synthesis or hydrolysis, ATP quantity and/or function of ATP in cells via post-translational modification of an ATP synthase subunit and/or precursor thereof.

Another aspect of the present invention relates to compositions and methods for modulating post-translational modifications of an ATP synthase subunit and/or precursor thereof in cells, said compositions and methods being those which induce preconditioning.

Another aspect of the present invention relates to compositions and methods for preconditioning organs and preventing cell injury and/or cell death by regulating ATP synthesis or hydrolysis, ATP quantity and/or function of ATP in cells. Also provided are methods for identifying compositions and methods for preconditioning organs and preventing cell injury and/or cell death by determining the ability of the composition or method to modulate post-translational modifications of an ATP synthase subunit and/or precursor thereof in cells and/or to regulate ATP synthesis or hydrolysis, ATP quantity and/or function of ATP in cells.

Another aspect of the present invention relates to methods for diagnosing and/or monitoring ischemic or hypoxic conditions via monitoring of post-translational modification of an ATP synthase subunit and/or precursor thereof. In one embodiment, diagnosis of an acute ischemic or hypoxic condition in a subject is performed by comparing levels of a post-translationally modified ATP synthase subunit and/or precursor thereof measured in the subject with levels of the post-translationally modified ATP synthase subunit and/or precursor in a control. An increase in levels of a post-translationally modified ATP synthase subunit and/or precursor thereof in the subject as compared to the control is indicative of an acute ischemic or hypoxic condition in the subject. In another embodiment, the present invention provides a method for differentiating between chronic ischemic or hypoxic tissue injury and acute ischemic or hypoxic tissue injury in a subject based upon detection of a post-translationally modified ATP synthase subunit and/or precursor thereof in a sample from the subject, wherein acute injury is characterized by a greater quantity of said post-translationally modified protein.

### BRIEF DESCRIPTION OF THE FIGURES

FIG. 1 is a schematic representation of mitochondrial ATP synthase of yeast including all the identified subunits. The yeast mitochondrial ATP synthase resembles mammalian mitochondrial ATP synthase and is representative of mitochondrial ATP synthases in general. As shown herein, the beta subunit together with the alpha subunit and the OSCP subunit make up the F₁ subunit, which is connected by a stalk to the Fₒ subunit. Many other protein subunits, encoded by both mitochondrial and nuclear DNA, are also part of the ATP synthase complex.

FIGS. 2A(1), 2A(2), 2B(1) and 2B(2) show an enlargement of a silver stain of a 2-dimensional gel of ATP synthase β chain precursor (molecular weight approximately 56.3-56.6 kDa; pI approximately 5.1 to 5.3) in control myocytes at protein loads of 100 μg (FIG. 2A(1)) and 250 μg (FIG. 2A(2)) and adenosine-treated (60 minutes at 100 μM) rabbit myocytes at protein loads of 100 μg (FIG. 2B(1)) and 250 μg (FIG. 2B(2)).

FIG. 3 shows an enlargement of a western blot from two-dimensional gel electrophoresis (molecular weight approximately 56.3-56.6 kDa; pI approximately 5.1 to 5.3) from FIG. 2 comparing isolated myocytes obtained from rabbit hearts that were either not treated (FIG. 3A) or treated with 100 μM adenosine for 60 minutes (FIG. 3B). This time period and concentration of adenosine are sufficient to protect against cell death. An anti-ATP synthase β-chain antibody was used for immunoblotting.

FIGS. 4A and 4B show mass spectra obtained by MALDI-TOF of tryptic in-gel digests of modified (FIG. 4A) and unmodified (FIG. 4B) ATP synthase β chain precursor.

FIGS. 5A, 5B, 5C and 5D show enlargements of composite images from two-dimensional silver-stained gels showing modifications to ATP synthase β-chain precursor in a chronic ischemic swine model (FIGS. 5A and 5B) and in an acute ischemic rabbit model (FIGS. 5C and 5D). FIG. 5A shows ATP synthase β-chain precursor in samples from sham-operated (control) swine that underwent the surgical procedure for occlusion of the mid-third of the left anterior descending coronary artery branch (LAD) but with no actual LAD occlusion (n=4). FIG. 5B shows ATP synthase β-chain precursor in samples from swine that underwent the same surgical procedure except with LAD occlusion (n=5). The LAD occlusion in these latter swine caused prolonged ischemia and led to chronic heart failure. No increase in post-translational modifications of the ATP synthase β-chain precursor relative to the sham-operated (control) swine was observed in myocardial tissue from these swine after 6 weeks of chronic injury. FIG. 5C shows ATP synthase β-chain precursor in untreated isolated rabbit myocytes (n=4), and FIG. 5D shows ATP synthase β-chain precursor in isolated rabbit myocytes treated with adenosine for 60 minutes (n=4). An increase in the post-translational modifications of ATP synthase β-chain precursor was observed in this acute ischemic injury model.

FIGS. 6A and 6B show a silver stained gel and a western blot, respectively, from the inner mitochondrial membrane of rat liver evidencing the presence of modified ATP synthase β chain protein ((molecular weight approximately 51.5-51.8 kDa; pI approximately 4.9 to 5.0) as spots 9, 10 and 11 (based on molecular weight of mature ATP synthase protein). ATP synthase β chain identity was confirmed by western blot and by MS analysis by MALDI.

FIGS. 7A, 7B and 7C show 2-dimensional silver-stained gels of ATP synthase β chain precursor detected in whole cell homogenate (FIG. 7A), cytoplasmic extract (FIG. 7B) and myofilament protein extract (FIG. 7C) from a single biopsy sample of the left ventricle of a representative human patient undergoing coronary artery bypass surgery.

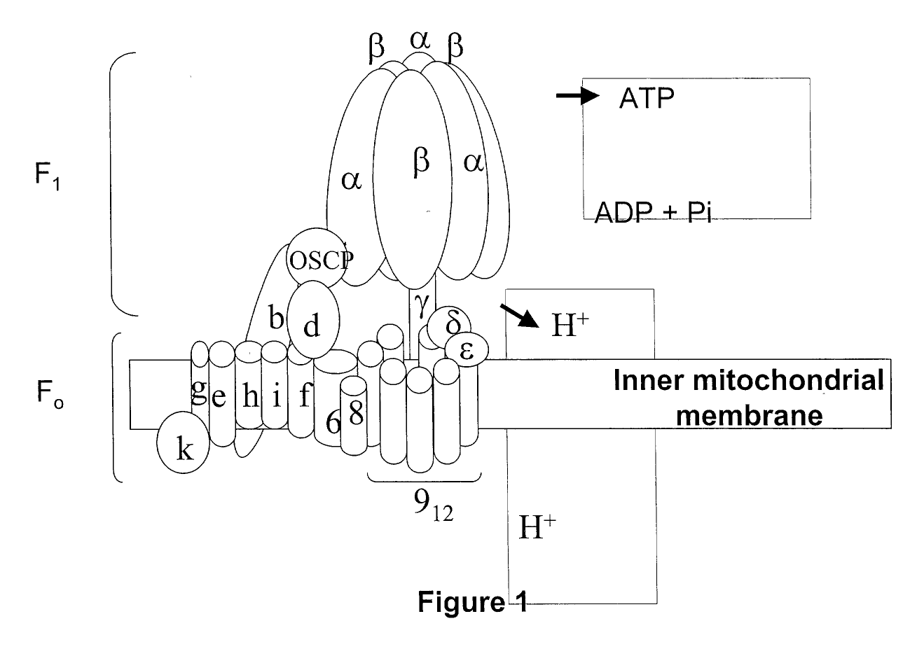

### DETAILED DESCRIPTION OF THE INVENTION

Preconditioning (PC), a phenomenon which exists in all species examined, including humans, is a form of protection wherein a brief ischemic or hypoxic episode prevents or reduces the extent of cellular or organ damage, death and/or dysfunction from subsequent prolonged ischemia. PC may also be recruited pharmacologically using an agonist such as adenosine. PC may also occur from other events and/or agents causing cell death, damage and/or dysfunction. Accordingly, the term “preconditioning” or “PC” as used herein is meant to be inclusive of ischemic, hypoxic, and/or pharmacological preconditioning, as well as preconditioning recruited by other events and/or agents causing cell death (necrosis or apoptosis), damage and/or dysfunction.

Preconditioning occurs in various organs and tissues including, but not limited to, myocardium, skeletal muscle, smooth muscle, liver, brain and kidney.

For example, adenosine is released from cells immediately with ischemia and affects both organs such as the heart as well as the vascular system through a second messenger signaling cascade triggered by binding to adenosine A₁, A₂ₐ, A_{2b} and/or A₃ receptors. In the heart, adenosine affects the intrinsic conducting system (bradycardia and AV block potential arrhythmia). In myocytes it affects the calcium current (negative inotropic) and mitochondrial K_{ATP} channels. It can affect the vascular system as well causing vasodilation. Adenosine causes preconditioning, potentially through activation of protein kinase C (PKC) and modulation of the mitochondrial and/or sarcolemmal K_{ATP} channel.

PC triggers two windows of protection, the first (classical PC) becoming manifest within 15 minutes and lasting 1-3 hours. The rapid onset and short duration of protection afforded by classical PC are likely the result of post-translational protein modifications, as 15 minutes is unlikely to be a sufficient time period to recruit significant de novo transcription and translation. A second, less effective window begins after 24 hours and lasts 24 to 72 hours. The second window is likely due to the presence of reactive oxygen species, novel protein synthesis produced by changes in gene regulation and/or expression, and post-translational modifications. Regulation of protein processing and/or turnover may also be responsible for modulation and/or alteration of nascent and/or functional protein quantities in this second window.

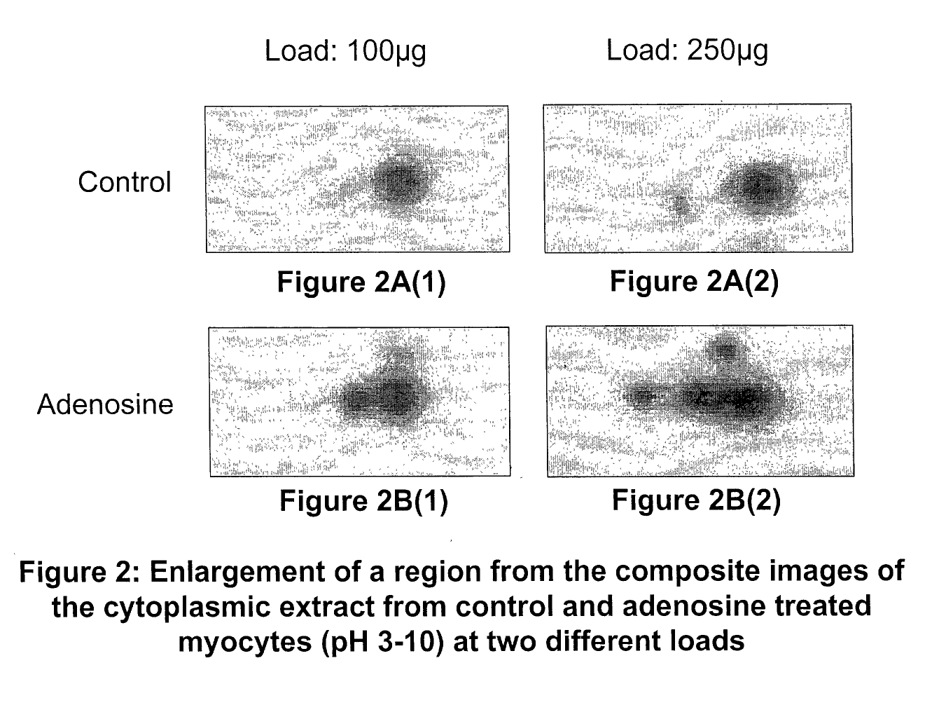

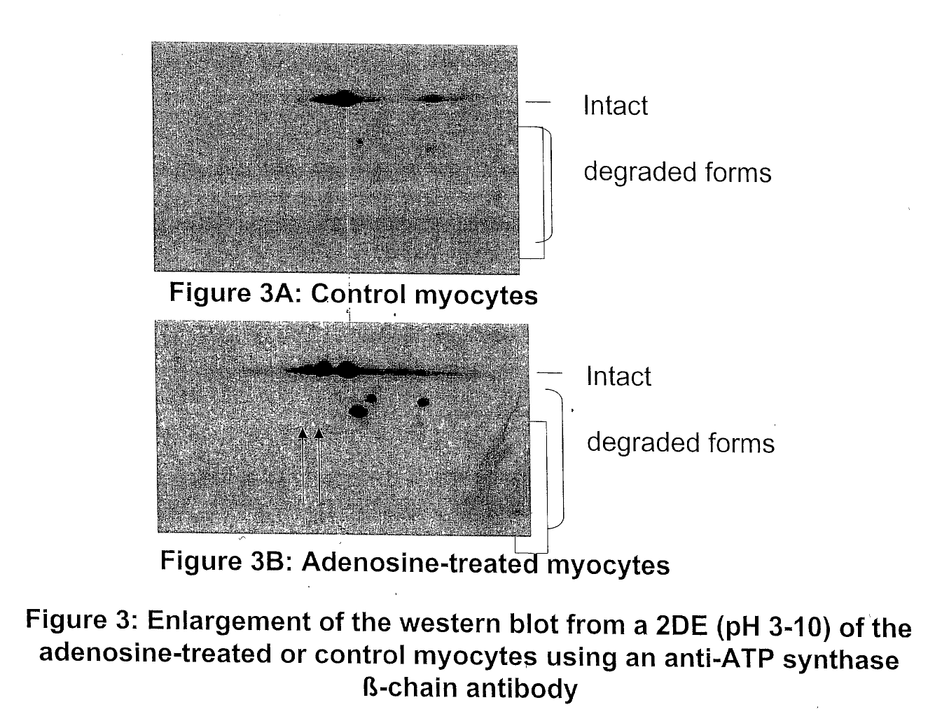

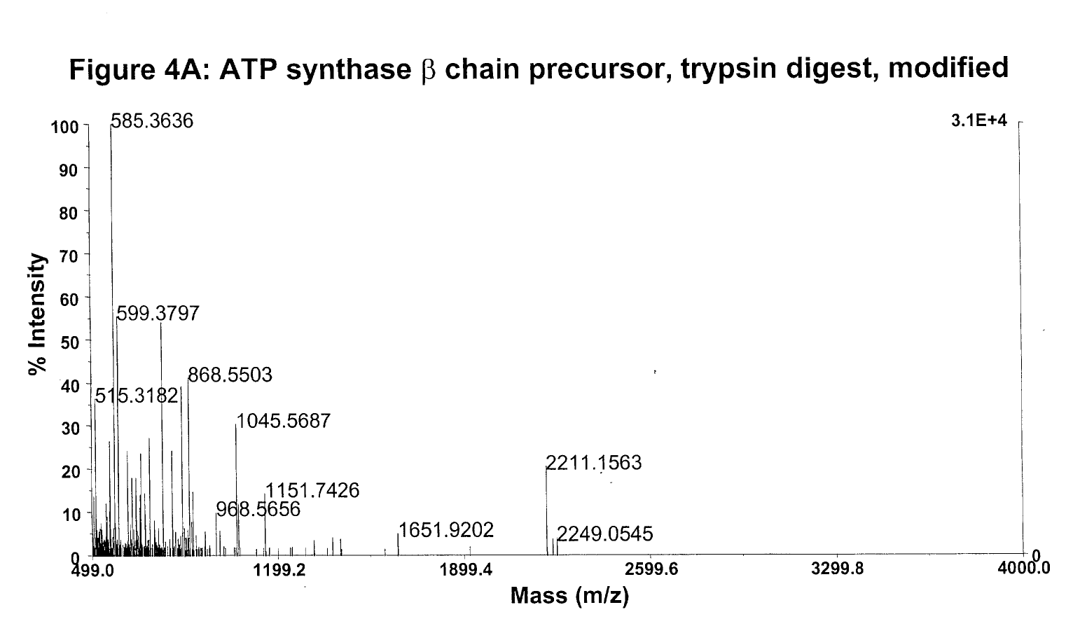

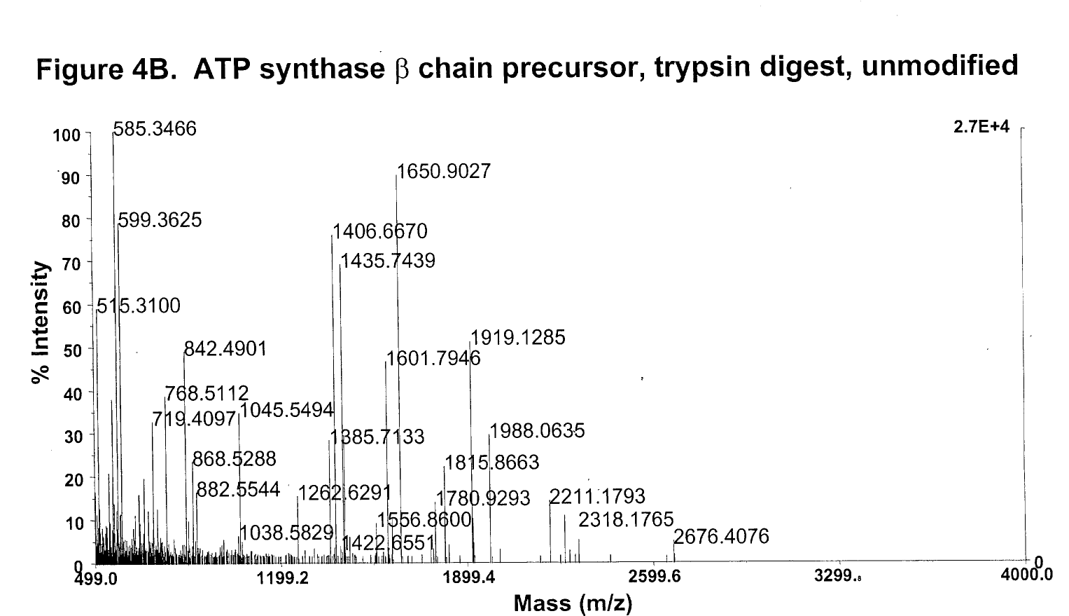

Two-dimensional gel electrophoresis analysis of the cytoplasmic extract of adenosine-treated isolated cardiomyocytes (n=4) at concentrations capable of invoking preconditioning has now revealed modifications of the mitochondrial ATP synthase β chain precursor. Two additional spots at the molecular weight of the intact protein, but which are more acidic, are present in adenosine-treated myocytes subjected to isoelectric focusing in the first dimension and SDS-PAGE in the second dimension, followed by silver stain (see FIGS. 2B(1) and 2B(2)) or western blot analysis using an ATP synthase β-chain antibody (see FIG. 3B). In contrast, control (untreated; FIGS. 2A(1), 2A(2) and 3A) cytoplasmic extracts had only a single protein spot.

The additional spots represent post-translationally modified mitochondrial ATP synthase β chain precursor. These two modified forms of ATP synthase β chain precursor are produced upon adenosine-invoked preconditioning.

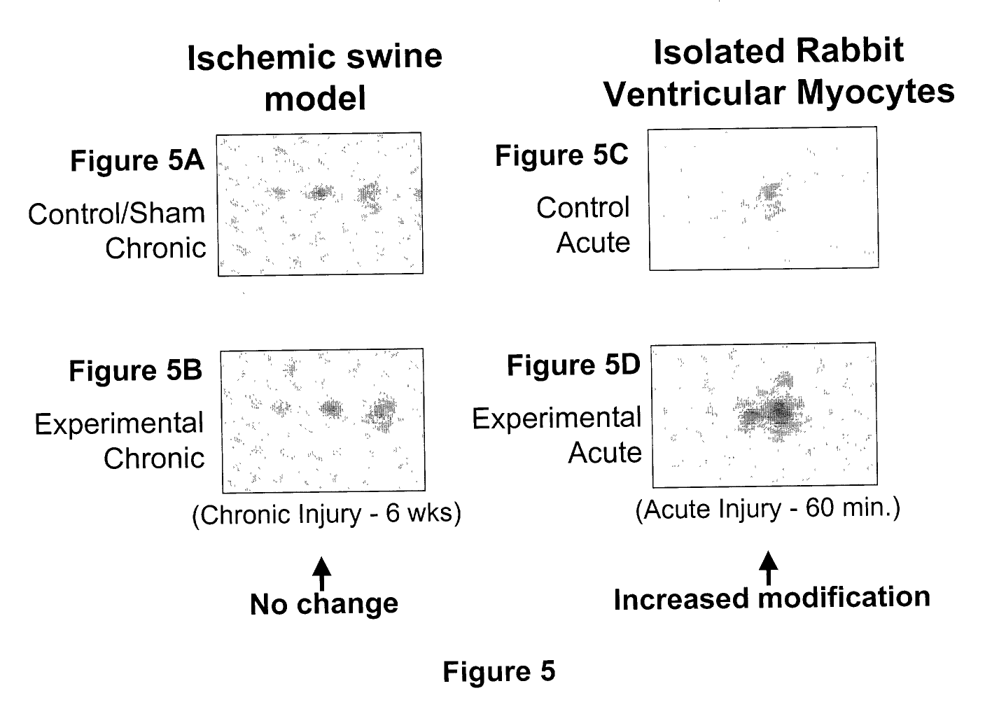

In initial experiments, modifications to the ATP synthase β chain precursor were observed in acute but not chronic cardiac injury. Protein profiles were determined in two chronic cardiac injury models. Specifically, protein profiles were determined in cardiac samples from an ischemic swine model after 6 weeks of injury. FIG. 5A shows the protein profile of sham-operated swine that underwent a surgical procedure for occlusion of the mid-third of the left anterior descending branch of coronary artery (LAD) with no occlusion. FIG. 5B shows ATP synthase β-chain precursor in samples from swine that underwent the same surgical procedure with LAD occlusion. No increase in modifications was observed in myocardial tissue from these swine after 6 weeks of chronic ischemic injury. Similar results were observed in a transgenic mouse model of chronic cardiac injury, the RAC1 mouse, which expresses constitutively active monomeric G protein causing lethal hypertrophy or remodeling within 18 days after birth. In contrast, as shown in FIGS. 5C and 5D acute ischemic cardiac injury caused modification to the ATP synthase β-chain precursor. FIG. 5C shows ATP synthase β-chain precursor in untreated isolated rabbit myocytes (n=4) and FIG. 5D shows ATP synthase β-chain precursor in isolated rabbit myocytes treated with adenosine for 60 minutes (n=4). Post-translational modifications to the ATP synthase β-chain precursor were detected in the adenosine-treated myocytes. Thus, as shown by these experiments, the presence or absence of post-translational modifications of ATP synthase or a precursor thereof, as well as comparison of different quantities of such species, can be used as a means for distinguishing between chronic and acute ischemic tissue injury. Further, the presence of post-translationally modified ATP synthase subunit and/or precursors thereof in acute ischemic injury can be used in the design of new treatments for acute as well as chronic ischemic tissue injury.

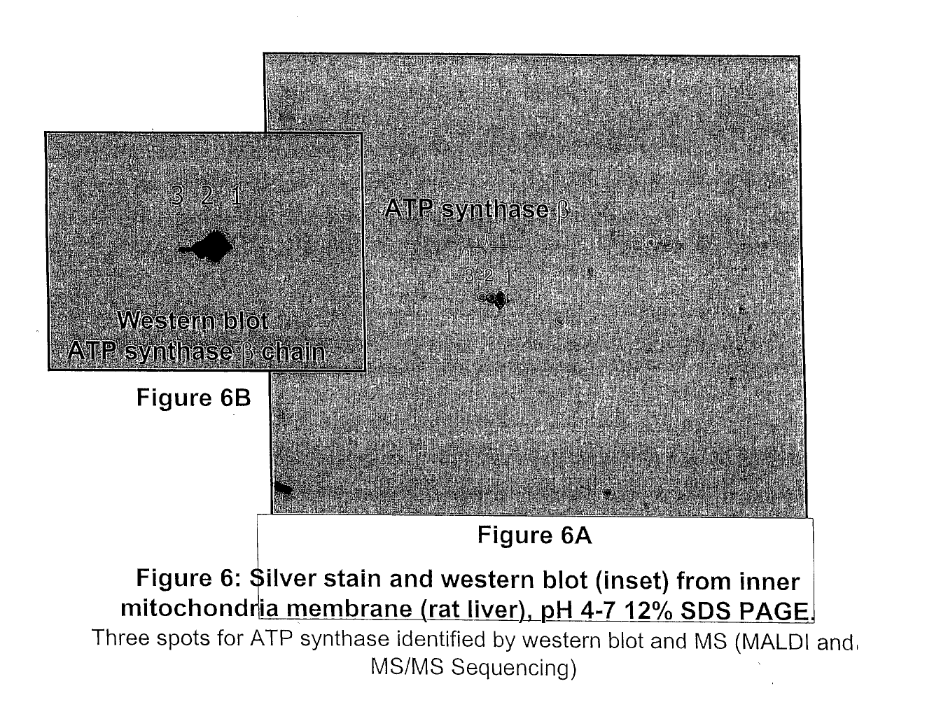

The presence of post-translationally modified ATP synthase has also been demonstrated in mitochondria, in particular the inner mammalian mitochondrial membrane. As shown in FIG. 6, modified forms of ATP synthase β chain, as shown by three spots, were observed by silver stain and corresponding western blot of a sample of inner mitochondrial membrane prepared from rat liver. Mass spectrometry confirmed spots 9, 10 and 11 all to be ATP synthase β chain. Thus, as shown herein, post-translational modifications occur in both ATP synthase β chain precursor and mature mitochondrial ATP synthase β chain when part of the ATP complex. Such post-translational modifications, particularly of the mature form, could affect ATP production directly or indirectly.

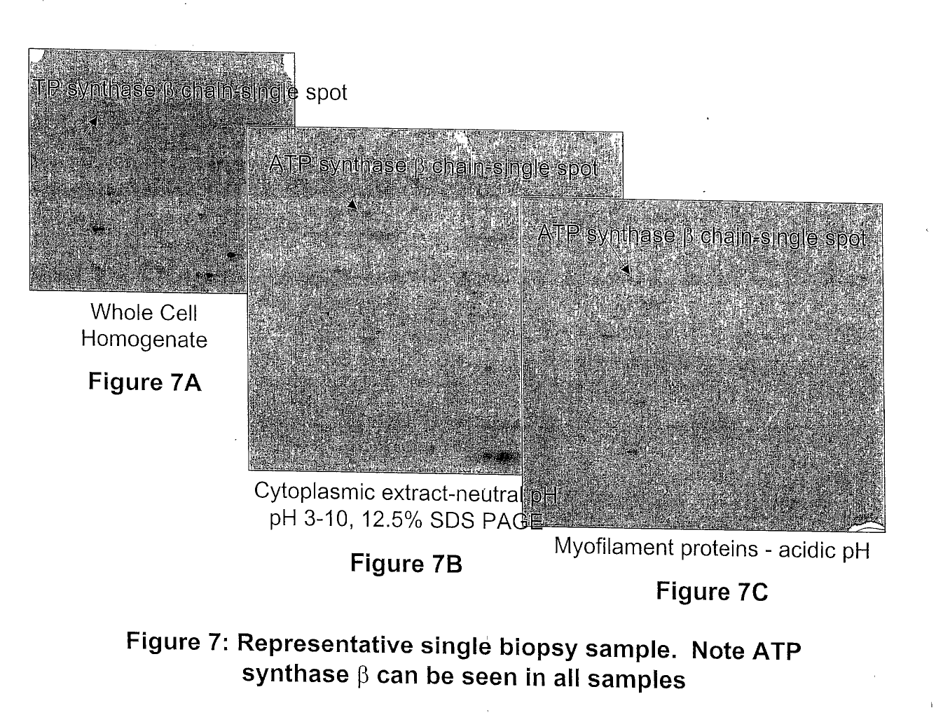

ATP synthase β chain is also detectable in human cardiac tissue. As shown in FIGS. 7A through 7C, ATP synthase β chain was detected via silver stain in whole cell homogenate, cytoplasmic extract and myofilament proteins prepared from a single biopsy sample obtained from the left ventricle of a human patient undergoing coronary artery bypass surgery.

For purposes of the present invention, by “post-translationally modified” it is meant to be inclusive not only of phosphorylation of amino acid residues, but also of other chemical adducts. Chemical adducts known in the art relating to post-translational modification of proteins include, but are not limited to, phosphorylation, glycosylation, glycation, myristylation, prenylation, phenylation, acetylation, nitrosylation, oxidation, s-glutathiolation, amidation, biotinylation, c-mannosylation, flavinylation, farnesylation, formylation, geranyl-geranylation, hydroxylation, lipoylation, methylation, palmitoylation, sulphation, gamma-carboxyglutamic acids, N-acyl diglyceride (tripalmitate), O-GlcNAc, pyridoxal phosphate, phospho-pantetheine, and pyrrolidone carboxylic acid. Preferred chemical adducts are phosphorylation, oxidation, glycosylation, myristylation, prenylation, acetylation, nitrosylation, and sulphation. Thus, by “post-translationally modified” it is meant to be inclusive of any of the above chemical adducts and/or any combination thereof. As shown herein post-translational modifications of a precursor of an ATP synthase subunit may occur, as well as of a mature form of an ATP synthase subunit.

It is believed that post-translational modification of the ATP synthase β chain precursor, as well as post-translational modifications of a mature form of ATP synthase or subunits or other precursors thereof, represents a unique mechanism for control of ATP synthesis or hydrolysis, ATP synthase function and/or quantities of ATP in the cell by controlling the amount of complex formed and present in the mitochondria. Thus, increased post-translational modifications due to adenosine treatment are believed to alter the amount of ATP synthase protein complex in the mitochondria over time. Post-translational modification of an ATP synthase subunit and/or precursor thereof, in particular the ATP synthase β chain and its precursor(s), may alter incorporation into the inner mitochondrial membrane (e.g., by changing the affinity of an ATP synthase precursor for proteolytic enzymes or for other ATP synthase subunits) directly or through cell localization such as via a scaffolding protein or by targeting of the protein to the mitochondrial membrane itself.

Alternatively, post-translational modifications to an ATP synthase subunit and/or precursor thereof may affect efficiency and function of the ATP synthase portion of the mature F₁Fₒ complex. This may involve alterations to normal substrate affinity (e.g., affinity for ADP, Pi, H⁺, or ATP) or for other subunits, or influence the efficiency of interactions between ATP synthase and potential regulators of ATP synthase during hypoxia or ischemia, therefore modulating ability of the mature complex to assemble/degrade/turnover ATP. In particular, it is known that the reduction of pH within the mitochondrial matrix that occurs during ischemia activates an ATP synthase inhibitor protein. This protein, known as IF₁, has been shown to interact with the ATP synthase β chain in the mature ATP synthase complex during ischemia, thereby preventing rotation of the F₁ portion and reducing the rate of ATP consumption. The IF₁ protein is an example of a such a protein whose interactions with ATP synthase may be influenced by post-translational modifications.

By promoting or inhibiting post-translational modifications of mitochondrial ATP synthase subunit and/or precursors thereof, ATP being synthesized and/or hydrolyzed in the cell can be regulated. Thus, one aspect of the present invention relates to compositions and methods for modulating amounts of ATP synthase and/or ATP, ADP, inorganic phosphate (Pi) and/or hydrogen (H⁺) ions in cells. By “compositions”, as used herein, it is meant to encompass any chemical or biological agent, including, but not limited to pharmacological agents, which modulates post-translational modification of a mitochondrial ATP synthase subunit and/or precursors thereof, ATP being synthesized and/or hydrolyzed in the cell. By “modulating” it is meant an increase or decrease in the net gain of ATP by increasing or decreasing amounts of ATP synthase and/or ATP synthase activity and/or ATP, ADP, Pi or H⁺ ions, and/or an increase or decrease in ATP synthesis or hydrolysis in the cells exposed to compositions or methods which modulate ATP, as compared to cells not exposed to the same compositions and/or methods. For example, modulation of ATP in cells can be achieved through changing the amount of the component(s) of the cytoplasmic protein pool available for incorporation into the ATP synthase complex in the mitochondria or changing the substrate affinity of one or more components of the mature complex. As exemplified herein, altering via preconditioning a mitochondrial ATP synthase precursor, in particular the ATP synthase β chain precursor, modulates ATP in cells. As will be understood by those of skill in the art upon reading this disclosure, the concept of regulating ATP levels of the cell through control of the availability or affinity of specific ATP synthase components is also applicable to other components of this complex, as well as to chaperones and to other proteins involved in the assembly or degradation of this complex. For example, controlling levels of ATP synthase itself, as well other precursors and/or subunits such as the α-chain is also expected to be useful in modulating ATP in cells.

In one embodiment of this aspect of the present invention, compositions and methods or events for modulating ATP synthase and/or ATP, ADP, Pi and H⁺ ion amounts and/or ATP synthesis or hydrolysis in cells are the same as those compositions and/or methods or events which induce preconditioning of organs such as the heart, skeletal muscle, smooth muscle, brain, kidney and/or liver.

In another embodiment of this aspect of the present invention, new compositions and methods or events useful in modulating ATP synthase and/or ATP, ADP, Pi or H⁺ ion amounts or ATP synthesis or hydrolysis and/or in inducing preconditioning of organs can be routinely identified in accordance with the teachings herein. Compositions and/or methods or events which are demonstrated to modulate phosphorylation and/or other modifications of ATP synthase, subunits or precursors thereof, such as the β chain and its precursor(s), in accordance with assays described herein are expected to be useful in modulation of amounts of ATP, ADP, Pi or H⁺ ions and/or ATP synthase amount or activity and/or ATP synthesis or hydrolysis and in inducing preconditioning in organs.

Another aspect of the present invention relates to regulation of ATP synthesis or hydrolysis in cells and its roles in preconditioning and cell injury and/or cell death. Understanding the effects of post-translational modifications of ATP synthase subunits or precursors thereof, such as the ATP synthase β chain and its precursor, upon ATP synthesis or hydrolysis will lead to better treatment of patients suffering from cell injury or cell death such as that caused by ischemia-reperfusion injury. For example, following cardiac arrest during surgery there are little or no free nucleotides left in myocytes and acidity (hydrogen content) of the cells is increased. In some cases, adenosine is added to stimulate ATP synthesis. This may only aid in the short term if adenosine also causes a reduction in the quantity of the functioning F₁Fₒ ATPase in the mitochondria with time (time being required for the modified β chain to be incorporated into the mature complex). Long term treatment thus may require blocking or eliminating adenosine action subsequent to obtaining its beneficial short term effects. Alternatively, if post-translational modifications are demonstrated to be beneficial in that they increase processing or mitochondrial membrane localization of the precursor or enhance the enzymatic activity of the ATP synthase complex (e.g., reduce hydrolysis of ATP during ischemia by, for example, increasing interaction with IF₁), then further promotion of the modifications via administration of additional adenosine (or equivalent agent) may be desired.

Another aspect of the present invention relates to methods for diagnosing and/or monitoring in a subject preconditioning and/or ischemic or hypoxic conditions and/or the ability of cells or organs to survive injury by monitoring post-translational modifications of an ATP synthase subunit and/or precursor thereof. Post-translationally modified ATP synthase subunits and/or precursors may be detected in a sample of injured tissue as well as in a biological fluid such as blood, serum, plasma, urine or cerebrospinal fluid, obtained from the subject.

In one embodiment of this aspect of the present invention, levels of post-translationally modified ATP synthase or subunits or precursors thereof can be monitored in a subject to assess whether the organ has been subjected to sufficient preconditioning or requires additional preconditioning for protection from cell or organ injury or death.

In another embodiment, acute ischemic or hypoxic conditions can be distinguished or differentiated from chronic ischemic or hypoxic conditions by detection of a post-translationally modified ATP synthase subunit and/or precursor thereof. As shown herein, the presence of a modified ATP synthase subunit and/or precursor thereof is primarily observed in acute ischemic tissue injury. Accordingly, the presence (or increased amount) of a modified ATP synthase subunit and/or precursor thereof is indicative of an acute ischemic or hypoxic injury.

The discovery that post-translationally modified ATP synthase subunits and/or precursors thereof are present or increased in acute ischemic injury can also be used in the design and selection of compositions and methods or events for use in treatment of acute as well as chronic ischemic tissue injury. Compositions and methods or events are preferably designed or selected to increase or promote post-translational modifications of an ATP synthase subunit and/or precursor thereof.

For purposes of the present invention by “acute” ischemic or hypoxic injury it is meant injury resulting from any brief ischemic/hypoxic period (e.g., 30 seconds to 2 days) such as stunning, or pre-conditioning such as infarction (e.g., myocardial infarction (MI)), unstable angina and the like as well as brief exposure to other events or agents that cause or promote cell death, necrosis or apoptosis. In some cases, such as in stunning, acute injury may be reversible.

By “chronic” injury it is meant the injury resulting from longer ischemic/hypoxic episodes (e.g., durations of days to years), such as heart failure (HF) and diabetes or longer exposure to other events or agents that cause or promote cell death, necrosis or apoptosis. Chronic muscle injury includes situations where muscle injury (e.g., due to necrosis or apoptosis and loss of muscle cells) causes the muscle to have to compensate for loss of functioning muscle cells. This leads to hypertrophy or atrophy of the muscle.

Diagnosis of an ischemic or hypoxic condition can also be performed by comparing levels of a post-translationally modified ATP synthase subunit and/or precursor thereof measured in a subject with levels of the post-translationally modified ATP synthase subunit and/or precursor thereof in a control. An increase in levels of a post-translationally modified ATP synthase subunit and/or precursor thereof in the subject as compared to the control is indicative of an ischemic or hypoxic condition in the subject.

As used herein, by “control” it is meant, a sample obtained from an individual known not have an ischemic or hypoxic condition, a sample obtained previously from the subject prior to the onset or suspicion of the ischemic or hypoxic condition, or a standard from data obtained from a data bank corresponding to currently accepted normal levels of the post-translationally modified ATP synthase subunit and/or precursor thereof. Increased levels of the post-translationally modified ATP synthase subunit or precursor in the sample obtained from the subject as compared to levels in the control are indicative of the subject having an ischemic or hypoxic condition. The comparison performed may be a straight-forward comparison, such as a ratio, or it may involve weighting of one or more of the measures relative to, for example, their importance to the particular situation under consideration. The comparison may also involve subjecting the measurement data to any appropriate statistical analysis.

The following nonlimiting examples are provided to further illustrate the present invention.

### EXAMPLES

### Example 1

Isolation and Preconditioning of Rabbit Ventricular Myocytes

Ventricular myocytes from New Zealand White rabbits (weighing 1 to 2 kg) were isolated by collagenase dissociation, as described previously by Liu et al. (Circ. Res. 1996 78:443-454). Hearts were excised, then perfused with collagenase (1.0 mg/mL, Worthington type II) for 14 minutes at a maintained perfusion pressure of 75 mm Hg on a Langendorff apparatus, yielding >50% Ca²⁺-tolerant ventricular myocytes. Cell isolation was followed directly by pharmacological preconditioning, which was carried out by treatment with 100 μmol/L adenosine (Sigma) for 60 minutes in a 37° C. water bath, as described previously by Liu et al. supra. Untreated cells were prepared concurrently as drug-free controls. Equivalent 25 μL aliquots of cells (containing ˜30 mg/mL of protein as determined by Lowry assay (Lowry, O. H. J. Biol. Chem. 1951 193:265-275) were frozen and stored at −80° C. until analysis.

### Example 2

Protein Extraction and Subcellular Fractionation

All steps in the “IN Sequence” protein extraction protocol to produce cytoplasmic and myofilament enriched extracts were carried out at 4° C., and all centrifugations were conducted at 16000×g for 2 minutes at 4° C. Myocyte proteins were first extracted by two rounds of homogenization in 100 μL of HEPES extraction buffer, consisting of (in mmol/L) HEPES 25 (pH 7.4), NaF 50, Na₃VO₄ 0.25, phenylmethylsulfonyl fluoride 0.25, EDTA 0.5, and (in μmol/L) leupeptin 1.25, pepstatin A 1.25. Following homogenization and centrifugation, the supernatants were pooled and saved as the cytosolic extract. The remaining pellet was subjected to further extraction by two rounds of homogenization in 50 μL of acid extraction buffer, consisting of 1% v/v trifluoroacetic acid (TFA) and 1 mmol/L Tris (2-carboxyethylphosphine) hydrochloride (pH ˜2.0). Supernatants were again pooled, and saved as the acid extract. The two extracts and remaining pellet were frozen and stored at −80° C.

### Example 3

Two-Dimensional Gel Electrophoresis (2-DE)

Isoelectric focusing (IEF) of cytoplasmic extract (loaded at 100 or 250 μg per gel) was carried out using a Protean® IEF cell (Bio-Rad) according to the manufacturer's protocol. Immobilized pH gradient (IPG) Ready Strips™ (170 mm pH 4-7 or pH 3-10 linear gradient, Bio-Rad) were actively rehydrated at 50 volts (V) for 10 hours to enhance protein uptake, then subjected to the following conditions using a rapid voltage ramping method: 100 V for 25 Volt-hours (Vh), 500 V for 125 Vh, 1000 V for 250 Vh, and 8000 V for 85 kVh. A Peltier temperature control platform maintained gels at 20° C. throughout IEF. Focused gels were stored at −20° C. prior to sodium dodecyl sulfate-polyacrylamide gel electrophoresis (SDS-PAGE).

For SDS-PAGE, IPG strips were incubated for 10 minutes in equilibration buffer (50 mmol/L Tris-HCl, pH 8.8, 6 mol/L urea, 30% v/v glycerol, 2% w/v SDS) supplemented with 10 mg/mL DTT, followed by a 10 minute incubation in equilibration buffer supplemented with 25 mg/mL iodoacetamide, then rinsed once with SDS-PAGE buffer (25 mmol/L Tris, 192 mmol/L glycine, pH 8.3, 0.1% w/v SDS). IEF strips were then embedded in a 5% acrylamide stacking gel and the proteins were resolved by 12.5% SDS-PAGE using a Protean® II XL system (Bio-Rad). Electrophoresis was carried out at 50 V for 30 minutes, followed by 150 V for 7.5 hours.

### Example 4

Protein Transfer and Western Blotting

Following 2-DE, gels were equilibrated in SDS-PAGE buffer supplemented to 20% v/v methanol for 10 minutes, then transferred in the same buffer to nitrocellulose at 200 mA constant current for 2 hours. Nitrocellulose membranes were then rinsed with phosphate-buffered saline/Tween-20 (PBS/T), consisting of (in mmol/L) NaCl 137, KCl 2.7, Na₂HPO₄ 10.1, KH₂PO₄ 1.8, pH 7.4 supplemented to 0.1% v/v Tween-20, then blocked overnight at 4° C. with 1% v/v blocking reagent (Roche Diagnostics) in PBS/T. Western blotting for ATP synthase β chain was performed at 1 μg/mL with the anti-ATP synthase β-chain antibody Clone No. 7E3-F2 (Molecular Probes Cat. No. A-21299, Eugene, Oreg.), and detected by chemiluminescence with an alkaline phosphatase-conjugated secondary antibody.

### Example 5

Silver Staining of Two-Dimensional Gels

Two-dimensional gels were silver stained according to the protocol of Shevchenko et al. (Anal. Chem. 1996 68:850-858) for compatibility with subsequent analysis of protein by mass spectrometry. Gels were fixed overnight in 50% v/v methanol, 5% v/v acetic acid, followed by 50% v/v methanol for 10 minutes, then 10 minutes in deionized distilled (dd) H₂O. Gels were sensitized for 1 minute in 0.02% w/v sodium thiosulfate, followed by two 1-minute ddH₂O washes, then incubated in chilled (4° C.) 0.1% w/v silver nitrate for 20 minutes, followed again by two 1-minute ddH₂O washes. Proteins were then visualized by several washes with developing solution (2% w/v sodium carbonate, 0.04% v/v formalin) until a desired level of staining was achieved, after which development was stopped with 5% v/v acetic acid.

### Example 6

Image Analysis and Quantification

Silver-stained 2-D gels were digitized at 150 dpi (pixels per inch) resolution using a PowerLookII® scanner (UMAX® Data Systems, Inc.) on a Sun® Ultra5™ computer (Sun Microsystems, Inc.). Protein spots were then located, quantified, and matched to spots on other gels using Investigator™ HT Proteome Analyzer 1.0.1 software (Genomic Solutions, Inc.). Fifteen manually defined spots were selected as anchors for triangulation of remaining spots. Composite images were then prepared by matching spots from four gel images for each treatment group (adenosine and control), and normalized using a match ratio method to compensate for any variation in protein loading and level of silver stain development between gels.

### Example 7

Mass Spectrometry

Protein spots extracted from 2-D gels were destained according to Gharahdaghi et al. (Electrophoresis 1999 20:601-605), then dried under vacuum before enzymatic digestion with sequence-grade modified trypsin (Promega) or ASP N (Sigma). Peptides were extracted with 50% acetonitrile (ACN)/5% TFA, dried under vacuum, and reconstituted with 3 μL of 50% ACN/0.1% TFA. Reconstituted extract (0.5 μL) was mixed with 0.5 μL of matrix (10 mg/mL α-cyano-4-hydroxy-trans-cinnamic acid in 50% ACN, 0.1% TFA), spotted on a stainless steel 100-well mass spectrometry plate, and air-dried.

MALDI-TOF MS of Cytoplasmic Proteins:

Samples were analyzed using a Voyager® DE-Pro matrix-assisted laser desorption/ionization time-of-flight (MALDI-TOF) mass spectrometer (PerSeptive Biosystems) reflector equipped with a 337 nm nitrogen laser operated in the delayed extraction/reflector mode with an accelerating voltage of 20 kV, grid voltage setting of 72%, and a 50 ns delay. Five spectra (50-100 laser shots/spectrum) were obtained for each sample. External calibration was performed using a Sequazyme Peptide Mass Standard kit (PerSeptive Biosystems) containing the following standards: des-Arg-bradykinin, angiotensin-1, and Glu-fibrinopeptide B.

MALDI-TOF MS of Mitochondrial Proteins

MALDI MS spectra were collected on a Bruker Reflex III time-of-flight mass spectrometer (Bremen/Leipzig, Germany) equipped with a SCOUT 384 multiprobe inlet and a 337 nm nitrogen laser in positive ion mode with delayed extraction using the reflector option. Spectra were obtained by averaging 100-300 individual laser shots and then processed with the Bruker supporting software. The spectra were internally calibrated with trypsin autolysis peptide peaks and matrix peaks.

MALDI-QTOF MS/MS:

MALDI MS/MS spectra were collected on an Applied Biosystems/MDS-Sciex QSTAR pulsar QTOF instrument (Concord, Ontario, Canada) equipped with an orthogonal MALDI source employing a 337 nm nitrogen laser. The instrument was operated in positive mode and collision-induced dissociation (CID) of peptides was achieved with argon as the collision gas. Spectra were acquired and processed using Sciex support software.

### Example 8

Bioinformatic Data Analysis

Peptide mass fingerprinting was conducted with the database search tool MS-Fit in the program Protein Prospector (version 4.0.4), to search the Swiss-Prot. Jun. 26, 2002 protein database. A number of restrictions were applied to the search: species=mammals, pI range 4.5-5.5, mass range 40-60 kDa (50 ppm mass tolerance for peptides from the unmodified protein, and 100 ppm mass tolerance for peptides from the modified protein), with a minimum of 4 peptides to match, and a maximum of one missed tryptic cleavage, with possible modifications including Cys-carbamidomethylation, Met-oxidation, protein N-terminal acetylation, and acrylamide modified Cys.

### Example 9

Ischemia-Induced Failing Heart Model in Swine

Neutered male swine (13-34 kg) underwent open chest surgery for occlusion of the mid-third of the left anterior descending branch of coronary artery (LAD). Sham-operated swine (SHAM) underwent the same surgical procedure except the LAD was not occluded. During open chest surgery and at termination, animals were under general anesthesia (A preanesthetic, atropine followed by a combination of ketamine, midasolam and isoflurane, with anesthesia maintained with isoflurane). Upon recovery the animals received analgesics as needed. At 4 weeks, echocardiography was performed on conscious, mildly sedated animals. To estimate the left ventricle ejection fraction, echocardiographs were performed in the lateral position, left side of the swine down, using a Pie Medical 200 scanner equipped with a 5.0/7.5 MHz probe (Indianapolis, Ind., USA). At 6 weeks post surgery animals were sacrificed, the hearts were excised, immediately frozen in liquid nitrogen and stored at −80° C.

### Example 10

RAC1 Mouse Model

Rac1 transgenic mice were created as described in Sussman et al. J. Clin. Invest. 2000 105: 875-886. To produce the transgene expressed in these mice, full length rac1 cDNA having a glycine to valine codon change at position 12 (V12 rac1) was inserted downstream of the α-MHC promoter. This point mutation has previously been shown to yield an activated protein. The Rac1 transgenic mice displayed constitutive expression of rac1 specifically in the myocardium and developed dilated cardiomyopathy. The transgenic mice were bred (n=4) and their tissues compared to those of corresponding non-transgenic (NTG) mice (n=4). Hearts were isolated from 2-3 week old Rac1 transgenic mice displaying the dilated phenotype (ratio of heart-to-body weight ranges from approximately 14 to 17) as well as age-matched NTG mice (ratio of heart-to-body weight ranges from approximately 5 to 6) and immediately frozen in liquid nitrogen prior to proteomic analysis.

### Example 11

Inner Mitochondrial Membrane Preparation

Purified inner mitochondrial membrane vesicles were prepared from rat liver according to Pederson et al. (Methods in Cell Biology, 1978, vol 20, Chapter 26, 411-481) which includes the modifications described by Hackenbrock and Hammon (J. Biol. Chem. 1975, 250; 9185-97) to the original protocol by Chan et al. (J. Cell. Biol. 1970, 45; 291-305).

### Example 12

Preparation of Human Biopsy Sample

Myocardial biopsies (20-100 μg) were obtained from the left ventricular epicardium of patients undergoing coronary artery bypass surgery. The samples were obtained from an area remote to the visually underperfused muscle, with no visible or pericardial fat. The samples were frozen immediately in liquid nitrogen, and then stored at −80° C. until analysis. Biopsy samples were analyzed as a whole tissue homogenate, or fractionated using a known protocol which enriches for cytoplasmic and myofilament proteins (Arrell et al. Circ. Res. 2001 89:480-7). Whole tissue homogenates of single biopsies were prepared by manual homogenization in 400 μl of IPG rehydration buffer containing 8 M urea, 2.5 M thiourea, 4% CHAPS, 0.5% carrier ampholytes (pH 4-6.5 or 3.5-10, Sigma, St. Louis, Mo., USA), 2 mM EDTA, with subsequent addition of 40 μl of 2.5 M DTT (final concentration of approximately 250 mM) just prior to IEF. Fractionation of biopsies into extracts 1 and 2 (enriched for cytosolic and myofilament proteins, respectively) was performed on ice as follows. Individual biopsies were homogenized in 20 μl of 20 mM imidazole, pH 7.4, with the addition of protease, kinase, and phosphatase inhibitors (1 μM leupeptin, 1 μM pepstatin A, 0.36 μM aprotinin, 0.25 mM PMSF, 0.2 mM sodium vanadate, 50 mM sodium fluoride, 2 mM EDTA). Following a 10 minute centrifugation at 16000×g at 4° C., the supernatant was collected, and the step repeated with the combined supernatant comprising extract 1. The remaining pellet was then homogenized in 20 μl 0.5% TFA, with 1 mM Tris(2-carboxyethyl)phosphine hydrochloride followed by a 10 minute centrifugation at 16000×g. The supernatant was collected, and the step repeated, with the combined supernatant comprising extract 2. IPG rehydration buffer (400 μl) was added to entire extracts, with the addition of 40 μl of 2.5 M DTT (final concentration of approximately 250 mM) just prior to IEF. The entire extract or homogenate from a single biopsy was then loaded onto a single gel. While protein quantification was impossible due to the small size of the myocardial biopsies, the range in size of the samples was consistent enough (20-50 μg) that slight increases and decreases in the development time of silver staining produced consistent staining intensities.

What is claimed is:

1. A method for differentiating between chronic ischemic or hypoxic tissue injury and acute ischemic or hypoxic tissue injury comprising detecting or measuring the level of a post-translationally modified ATP synthase subunit or precursor thereof in a sample of a subject wherein the presence or increased level of a post-translationally modified ATP synthase or precursor thereof in the sample is indicative of acute ischemic tissue injury.

2. A method for diagnosing an ischemic or hypoxic condition in a subject comprising comparing levels of a post-translationally modified ATP synthase subunit or precursor thereof measured in the subject with levels of the post-translationally modified ATP synthase subunit or precursor thereof in a control, wherein an increase in levels of the post-translationally modified ATP synthase subunit or precursor thereof in the subject as compared to the control is indicative of an ischemic or hypoxic condition in the subject.

3. A method for identifying a composition or event for preconditioning an organ and preventing cell injury or cell death comprising determining the ability of the composition or event to modulate a post-translational modification of an ATP synthase subunit or a precursor thereof in cells or to regulate ATP synthesis or hydrolysis in cells.

4. The method of claim 3 wherein the ATP synthase subunit is ATP synthase β chain and the ATP synthase precursor is ATP synthase β chain precursor.
